(19) United States
(12) Patent Application Publication
(10) Pub. No.: US 2016/0085415 A1
(43) Pub. Date: Mar. 24, 2016

(54) MULTI-PARAMETER, RISK-BASED EARLY WARNING AND ALARM DECISION SUPPORT WITH PROGRESSIVE RISK PIE VISUALIZER

(71) Applicant: KONINKLIJKE PHILIPS N.V., EINDHOVEN (NL)

(72) Inventors: John Francis HUMPHRYS, Needham, MA (US); Youssef ABOU-HAWILI, Andover, MA (US)

(21) Appl. No.: 14/855,675

(22) Filed: Sep. 16, 2015

Related U.S. Application Data
(60) Provisional application No. 62/053,952, filed on Sep. 23, 2014.

(51) Int. Cl. G06F 3/0481; G06F 1/16; G06F 3/16; A61B 5/00

(52) U.S. Cl. CPC G06F 3/04817; A61B 5/7445; A61B 5/746; G06F 1/163; G06F 3/167

(57) ABSTRACT

A medical monitoring device includes a data processor and a display device communicatively connected to the data processor. The data processor constructs a circular icon representative of received monitored physiological and alarm data for a plurality of vital signs monitored for at least one patient, the circular icon divided into wedges and each wedge corresponds to one of the plurality of vital signs, and label each circular icon with an identity of one of the at least one patient, and the identity includes at least one of a patient name and a patient location. The display device displays the constructed circular icon and the identity for the at least one patient.

### CROSS REFERENCE TO RELATED APPLICATIONS

This application claims the benefit of U.S. provisional application Ser. No. 62/053,952 filed Sep. 23, 2014, which is incorporated herein by reference.

The following relates generally to medical monitoring of patients. It finds particular application in conjunction with medical monitoring alarms and healthcare practitioner notification, and will be described with particular reference thereto. However, it will be understood that it also finds application in other usage scenarios and is not necessarily limited to the aforementioned application.

Alarm fatigue is considered a national problem and the number one medical device technology hazard in 2012 according to the American Association of Medical Instrumentation. The problem of alarm desensitization is multifaceted and relates to high false alarm rates, poor positive predictive value, lack of alarm standardization, and the number of alarming medical devices in hospitals today. Hospitals commonly monitor various vital signs for each patient, such as respiratory rate (RR), non-invasive blood pressure (NBP), blood oxygen (SpO₂), and heart rate (HR). For example, whenever a vital sign reading exceeds an alarm limit, which is generally set by hospital protocol, an alarm is sounded. In response to an alarm, a healthcare practitioner goes to the patient, checks the patient's condition, takes appropriate action, and silences the alarm. When a single vital sign has exceeded the alarm limit, prompt action is often not necessary. Because all alarms appear and/or sound substantially the same, the healthcare practitioner is called to go to the patient to determine whether more than one alarm has been triggered.

Many alarm monitors are configured to put as much detail into available display space as possible in order to give a detailed picture to the healthcare practitioner or caregiver, which can be counter-productive for a caregiver called to decide whether to continue the task at hand or interrupt and proceed to the alarming patient. A typical caregiver in a hospital cares for up to 6 patients on daytime shifts and double or triple that amount on evening and night shifts. The patients are distributed about a floor. Efficiency calls for completing each task and proceeding to the next task in a specific order, often in another room or location to improve an overall level of care. Alarms change efficient processing of tasks by calling for service to the alarming patient, e.g. reordering of tasks, which can be inefficient.

Another issue for healthcare practitioners to accommodate is the device used to deliver the alarms. Wall mounted display screens in corridors showing vital signs for the patients on the floor or area are common. For healthcare practitioners, a wall mounted display provides detailed information including identity and location of an alarm displayed hands-free, but may call for travel to view. The detailed information can be more than the healthcare practitioner needs to make a decision, and can be inefficient to mentally process the detailed information. Furthermore, centrally mounted displays typically include patients that all the caregivers are assigned, which calls for searching through the displayed information for assigned patients. A portable device that is set-down in a hospital carries a risk of damage and/or contamination.

Another issue for healthcare practitioners is maintaining a picture of patient condition over time. For example, viewing vital sign conditions change or progress over time can alter a decision by a healthcare practitioner interrupting another task. Maintaining the mental picture is difficult with detailed numbers and voluminous data. However, as additional vital sign measures are alarmed for a patient, e.g. an increased RR alarm and followed by decreased NBP alarm, suggesting a declining condition, the urgency of service increases. Another alarmed patient may just have been restless, and the patient movement triggered a RR alarm, and no other vital signs alarm, which does not suggest an urgent condition.

The following discloses a new and improved multi-parameter, risk-based early warning and alarm decision support system with progressive risk pie visualizer, which addresses the above referenced issues, and others.

In accordance with one aspect, a medical monitoring device includes a data processor and a display device communicatively connected to the data processor. The data processor constructs a circular icon representative of received monitored physiological and alarm data for a plurality of vital signs monitored for at least one patient, the circular icon divided into wedges and each wedge corresponds to one of the plurality of vital signs, and label each circular icon with an identity of one of the at least one patient, and the identity includes at least one of a patient name and a patient location. The display device displays the constructed circular icon and the identity for the at least one patient.

In accordance with another aspect, a method of monitoring physiological and alarm data includes constructing a circular icon representative of received monitored physiological and alarm data for a plurality of vital signs monitored for at least one patient, the circular icon divided into wedges and each wedge corresponds to one of the plurality of vital signs, and labeling each circular icon with an identity of one of the at least one patient, and the identity includes at least one of a patient name and a patient location. The constructed circular icon and the identity for the at least one patient is displayed on a display device.

In accordance with another aspect, a medical monitoring device includes a wireless communication unit, a data processor communicatively connected to the wireless communication unit, and a display device communicatively connected to the data processor. The wireless communication unit receives the monitored physiological and alarm data from a central monitoring system. The data processor constructs a pie shaped representation of the received monitored physiological and alarm data for a plurality of vital signs monitored for at least one patient. The pie shaped representation is divided into wedges and each wedge corresponds to one of the plurality of vital signs. The data processor constructs a concentric circle in the pie shaped representation and the concentric circle includes an early warning score for the at least one patient and labels each circular icon with an identity of one of the at least one patient, and the identity includes a patient name and a patient location. The display device displays the constructed circular icon and the identity for the at least one patient.

One advantage is progressive risk visualization.

Another advantage resides in quickly understood and processed alarm information for decision making.

Another advantage resides in supporting hands free operation.

Still further advantages will be appreciated to those of ordinary skill in the art upon reading and understanding the following detailed description.

The invention may take form in various components and arrangements of components, and in various steps and arrangement of steps. The drawings are only for purposes of illustrating the preferred embodiments and are not to be construed as limiting the invention.

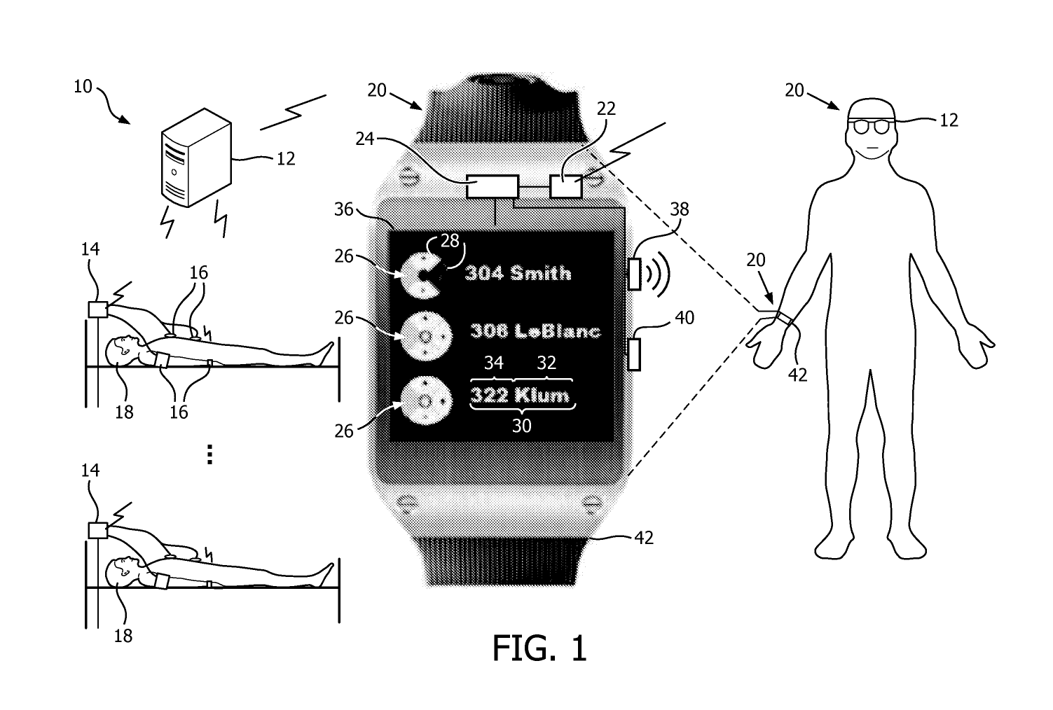

FIG. 1 schematically illustrates an embodiment of a multi-parameter, risk-based early warning and alarm decision support system with progressive risk pie visualizer for multiple patients on a web enabled watch.

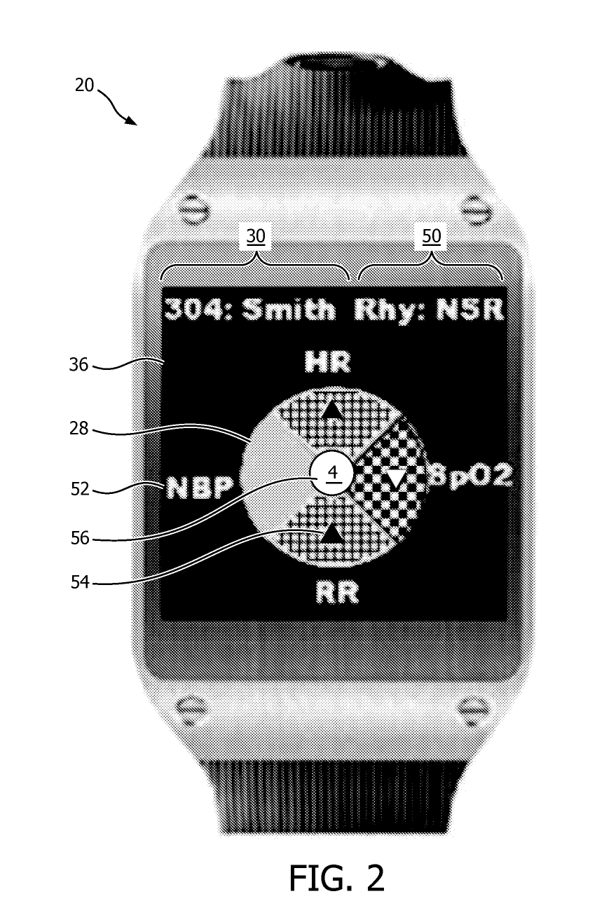

FIG. 2 illustrates an exemplary monitoring device display of the progressive risk pie visualizer for a single patient.

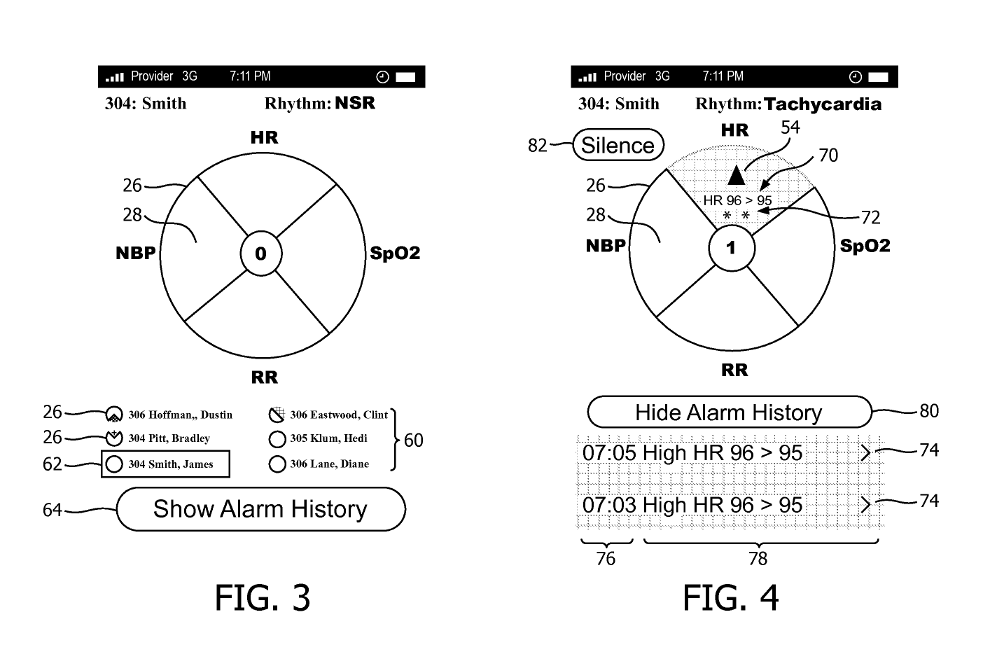

FIG. 3 illustrates an exemplary monitoring device display of the progressive risk pie visualizer for a clinician. It displays the data for one patient in a large format and uses smaller representations for the clinician's other assigned patients.

FIG. 4 illustrates an exemplary monitoring device display of the progressive risk pie visualizer for a patient with an alarm history.

In FIGS. 2-4 different hash patterns are used to represent various degrees of alarm severity. This severity could also be represented by colors such as yellow for medium severity and red for critical severity.

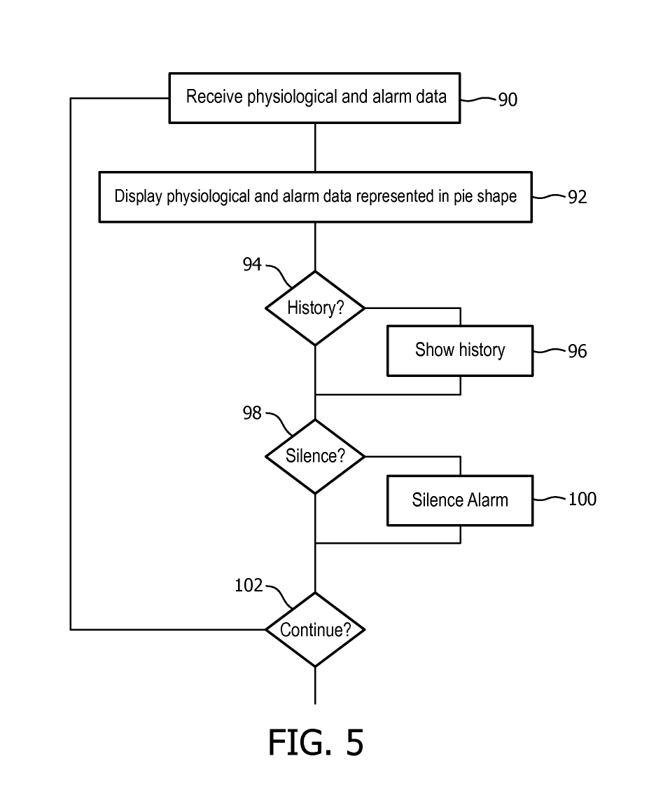

FIG. 5 flowcharts one method of monitoring physiological and alarm data to progressively visualize multi-parameter, risk-based early warning and alarms.

With reference to FIG. 1, an embodiment of a multi-parameter, risk-based early warning and alarm decision support system 10 with progressive risk pie visualizer is schematically illustrated. A central monitoring computing device 12 receives physiological data and alarm data from monitoring devices, telemetry systems, or other such devices 14. Each monitoring device 14 receives physiological data from one or more physiological sensors 16 to monitor one or more vital signs or measured parameters of a patient 18, such as non-invasive blood pressure (NBP), heart rate (HR), respiration rate (RR), blood oxygen (SpO2), and the like. The physiological sensors 16 sense the physiological signals and communicate the physiological signals to the monitoring device 14.

The monitoring device 14 receives the signals and processes the signals to determine values representative of the vital sign, e.g. values for HR, RR, etc. The signals are received through wired and/or wireless communications connections. The monitoring device 14 compares the determined values to one or more thresholds or alarm limits, e.g. upper and/or lower thresholds, to determine an alarm condition of each vital sign. For example, if the determined HR exceeds an upper predetermined value, a yellow alarm is determined for HR, or if the determined HR drops below a lower predetermined value, a red alarm is determined for HR. As another example, yellow can indicating exceeding an alarm limit and red can indicate exceeding the limit by a larger amount, such as 10%. Alarm conditions can use indicators, such as green for normal, yellow for a first threshold level, and red for a second threshold level. The monitoring device 14 of each patient 18 communicates the physiological monitored data, e.g. signals and/or values, and the alarm data to the central monitoring computing device 12. The alarm data can include the alarm indicator, the vital sign values triggering the alarm condition and the threshold values associated with the alarm condition. In addition, in some situations, values other than vital signs may be used. For example, for some patients lab values or electrolytes may be displayed.

The central monitoring computing device 12 receives the monitored physiological and alarm data from the monitoring device 14 of each patient 18, and determines an early warning score (EWS) for each patient 18 from the monitored data. In one embodiment, the monitoring device 14 determines the EWS. The EWS uses values, such as 0-5, which indicate a state of the health of the patient 18, e.g. 0 for normal, and 5 for critically ill. The monitored physiological and alarm data can be received through wired and/or wireless communication. The central monitoring computing device 12 is suitably embodied as a communications network connected server.

A medical monitoring device 20 includes a communications unit 22, which receives the monitored physiological and alarm data from the central monitoring computing device 12 for a selected set of patients according to assignment. For example, a healthcare practitioner is assigned care responsibilities for Smith in room 304, LeBlanc in room 306, and Klum in room 322, and medical monitoring device receives the monitored physiological and alarm data for Smith, LeBlanc, and Klum. The selection of the set of patients can be performed by the central monitoring computing device 12 and/or the medical monitoring device 20 from the set of all patients monitored by the central monitoring computing device 12. In one embodiment, the communications unit 22 includes a wireless communications component, such as a transceiver for cellular and/or data communications. In another embodiment, the communications unit 22 includes a wired communications component, such as a network interface card.

The medical monitoring device 20 includes a data processor 24, which constructs a circular or pie shaped icon 26 representative of received monitored physiological and alarm data for the vital signs monitored for the patient 18. The circular icon 26 is divided into wedges 28 and each wedge corresponds to one of vital signs or other measurement. Construction of the circular icon 26 can include selecting the icon components from storage and/or dynamically creating the circular icon 26 and/or the wedges 28. For example, a circular icon with default colored wedges is retrieved from storage, and the data processor overlays selected colored wedges according to alarm conditions different from the default. In another example, the circular is created through program instructions drawing the circle and filling with a default color, and the wedges are drawn through program instructions with the corresponding colors. The data processor 24 labels each circular icon with an identity 30 of the patient 18. In one embodiment the identity 30 includes a patient name 32 and a patient location 34.

A display device 36 communicatively connected to the data processor displays the constructed circular icon 26 and the identity 30 for the patient 18. The display device 36 can display the circular icon 26 and the identity 30 for assigned patients and/or selected sets of patients. As each vital sign for the patient 18 changes, e.g. change from normal to yellow or red alarm condition, the corresponding wedge 28 is changed to reflect the new alarm condition. The display of the pie shaped icon provides a progressive risk visualization. For example, visualization provides a quick view of each patient with the healthcare practitioner quickly scanning the pie shaped icons. As the wedges change indicators, e.g. more changed to yellow and/or red, the healthcare practitioner can readily understand the current and changed vital sign conditions, and relate an urgency of the overall condition of the patient to the task at hand, e.g. interrupt the task or defer. As a patient condition declines as evidenced through the vital sign data, the pie shape progressively increases in wedge formats suggestive of greater or progressive health risk, e.g. change over time with more area in the pie shape indicating alarm conditions.

The medical monitoring device 20 can include an audio output device 38, such as a speaker which generates audible indicators of alarm conditions and/or the EWS. The audible indicator can vary by tone, intensity, and/or pattern to indicate different alarm conditions and/or EWS scores.

The medical monitoring device 20 includes one or more input devices 40, such as a touch screen, microphone, keyboard, mouse, and the like. In one embodiment, the input devices include a touch screen and a microphone. The microphone receives voice responses for hands-free operation of the medical monitoring device 20.

In one embodiment, a frame assembly 42 mounts the data processor 24, the display device 26, and the communication unit 22, e.g. wireless, to be worn on a head or a wrist. For example, the frame assembly including a wrist band is worn by a healthcare practitioner on the wrist. The healthcare practitioner can view the display device 26 at a glance to view the alarm condition while performing other tasks, e.g. without interruption of a current task. In another example, as shown in FIG. 1, the frame assembly is worn as or in eye glasses and with display device in the peripheral vision of the wearer. The data processor 24 includes a non-transitory computer readable storage medium carrying instructions, e.g. software, which controls the data processor to construct the display and control the display device, the communications unit, and the input and output devices.

With reference to FIG. 2, an exemplary monitoring device display of the progressive risk pie visualizer for the patient is illustrated. The display includes the circular icon 26 labeled with the identity 30 of the patient. The display includes other key relevant information 50, such as heart rhythm, diabetes, which can be co-located with the relevant vital sign. For example, a normal sinus rhythm (NSR) is indicated. The key relevant information 50 is provided to indicate possible conditions or rule-out possible conditions, such as heart attack, stroke, and the like. The circular icon 26 includes one wedge 28 for each of the four vital signs, HR, NBL, RR, and SpO₂. Each edge 28 can include a label 52. The wedges 28 are located consistently, e.g. in the same location clockwise from patient to patient. For example, NBP is located at the left or 9:00 centered position, HR at the top or 12:00 centered position, SpO₂ on the right or 3:00 centered position, and RR at the bottom or 6:00 centered position. The centered positions can be different, e.g. 10:00, 1:00, 4:00, and 7:00. For more or less vital signs, the size and/or position of the wedges 28 are changed.

The format of each wedge 28 can vary by color, pattern, and/or intensity. The format of the wedge corresponds to the alarm condition. For example, the alarm conditions correspond to varying patterns or intensities with the colors of red, yellow, and green. The NBP wedge format is illustrated with a general gray pattern, and can be indicated in color with green indicating normal or no alarm condition. The HR and RR wedge formats are illustrated with a grid pattern, and can be indicated in color with yellow indicating a first alarm threshold exceeded. The SpO₂ wedge format is illustrated in a checkerboard pattern, and can be indicated in color with red indicating a second alarm threshold exceeded. Patterns rather than colors can be used to indicate the alarm state.

The format of the wedge 28 can include a single indicator 54, such as an arrow or triangular shape, located within or superimposed on the wedge 28 indicating a change in the vital sign, e.g. an increase or a decrease. For example, the HR formatted wedge includes a triangle with an apex pointed up or toward the top of the display indicating an elevated HR, and the SpO₂ includes a triangle with an apex pointed down or toward the bottom of the display indicating an lowered SpO₂.

The data processor 24 constructs in the center of the circular icon 26 a concentric circle 56, which includes the EWS for the patient 18. In the illustrated display, the EWS is represented with a number four in the concentric circle indicating a critical risk score. The EWS provides the healthcare practitioner with a single simple measure easily located concerning the progressive health risk for the patient 18. Locating the concentric circle 56 within the circular icon 26 provides faster and easier decision making by the healthcare practitioner than locating EWS separately, e.g. can be more readily related to the pie as a whole and the vital signs represented by the individual wedges. In one embodiment, the audio output device 38 sounds a tone, an intensity, a pattern, combinations, and the like indicative of the EWS for the patient 18.

In other embodiments, lab test values, such as BUN, creatine, electrolytes, and the like are also displayed. The lab values can be displayed as wedges, increasing the number of wedges if necessary. Alternately, the lab values can be displayed in the circle 56 instead of the EWS. As another option, the lab values can be displayed sequentially, e.g., with the EWS score in the circle 56. As another option, only lab results that violate a threshold are displayed with the appropriate background color or pattern.

With reference to FIG. 3, an exemplary monitoring device display of the progressive risk pie visualizer for a patient with assigned patients is illustrated. The display device includes a large viewable display, such as a handheld portable computing device, smartphone, and the like. The display includes circular icons 26 for assigned patients 60. In the display, the assigned patients 60 can be unordered or ordered according to the location, the name, and/or the EWS. Display space allocated to the assigned patients 60 can be scrollable. Each circular icon 26 is formatted according to the alarm condition of the corresponding patient 18. As shown two patients, Dustin Hoffman in 305 and Bradley Pitt in 304, each have two alarm conditions, or one wedge formatted indicating an alarm condition. Clint Eastwood in 306 is shown with two alarm conditions, or two wedges formatted indicating an alarm condition. James Smith in 304 is the active or selected patient 62, and is display in a larger format for faster review and comprehension by the healthcare practitioner. An input, e.g. voice command, touch of another patient identity, and the like can select a different patient for larger display.

Alarm history 64 is selectable for the selected patient 62. The alarm history selection is selected by input, such as by touch or by voice command.

With reference to FIG. 4, an exemplary monitoring device display of the progressive risk pie visualizer for a patient with an alarm history is illustrated. Smith in 304 is the selected patient 62. Illustrated is the single indicator 54 indicating an increasing vital sign of HR. The HR wedge is formatted with the triangle apex oriented up and includes the triggering alarm condition 70. The triggering alarm condition 70 can include the patient vital sign value, the alarm threshold value, and a relationship. For example, the value of 96 is the patient vital sign value, the value of 95 is the alarm threshold value, and the relationship is the greater than sign “>” expressed as “HR 96>95”, which provides a concise summary.

The formatted wedge can also include a secondary visual indicator 72, which reinforces the formatted color, pattern, or intensity of the wedge and/or aids color blind healthcare practitioners with an alternative mechanism to comprehend the formatted wedge. For example, a sequence of asterisks of a number corresponding to a threshold level indicates the alarm condition, e.g. one asterisk for a first threshold exceed, two asterisks for second threshold, and the like. The second visual indicator 72 can include different icons, shapes, symbols and the like.

An alarm history 74 is displayed. Each alarm history includes a time indicator 76 and a historical alarm condition 78. The alarm history is ordered by time. For example at a time of 07:05, the most recent alarm, a high HR value of 96 is greater than a threshold alarm value of 95. At a time of 7:03, an earlier alarm, a high HR value of 96 is greater than a threshold alarm value of 95. The alarm history can be hidden with a hide history input 80 such as a toggle button, voice command, touch command, and the like.

In response to one or alarm conditions for the patient, a silence input 82, such as a input button, voice command, touch command, the like, remotely silences the alarms. The silence input 82 can be site specific for operation. With voice command input of the medical monitoring device 20 worn on the body, the healthcare practitioner can remotely without travel, review the alarm conditions and silence the alarm condition in a hands free operation. The silence input 82 is received by the data processor 24 and can be communicated to the central monitoring computing device 12 and/or the monitoring device 14 using the communications unit 22. For example, an alarm condition is determined for Smith in room 304 while the healthcare practitioner is caring for Clint Eastwood in room 306. The HR pie wedge of Smith changes to a yellow color and the audio output is sounded. The healthcare practitioner sees the display and selects Smith with a voice command “select Smith” received by the microphone. Smith is displayed. The healthcare practitioner views an HR alarm wedge and sees the yellow color. The healthcare practitioner silences the alarm condition with a voice command “silence alarm”. The voice command is received through the microphone and the data processor formats a silence alarm command to the monitoring device.

With reference to FIG. 5, one method of monitoring physiological and alarm data to progressively visualize multi-parameter, risk-based early warning and alarms is flowcharted. In a step or by a module 90, physiological and alarm data is received from one or more monitoring devices 14, which monitor one or more vital signs of patients. The physiological and alarm data can be received by wired and/or wireless communications. For example, the physiological and alarm data can be received by wireless cellular and/or data communication from the central monitoring computing device or central monitoring system.

A circular icon representative of the received monitored physiological and alarm data is constructed in a step or by a module 92. The circular icon for each patient is divided into wedges and each wedge corresponds to one monitored vital sign, such as HR, RR, SpO₂, NBP and the like. Each circular icon is labeled with a patient identity and the identity includes a patient name and a patient location. The wedges are formatted according to the alarm conditions, which include colors, patterns and/or intensities. In one embodiment, the constructed circular icon includes the EWS as a concentric circle within the circular icon and an indicator value for the EWS. The constructed circular icon and the identity are displayed on the display device, which can be body worn, e.g. mounted on the frame assembly attached to the body of the healthcare practitioner. Alarm conditions can be sounded using the audio output device with a tone, pattern and/or intensity indicative of the EWS. For example, a pattern can repeat in shorter time intervals with greater intensity as the EWS values increase, e.g. greater patient risk score.

In a decision step or by a module 94, an input for alarm history is received and a display is determined. In a step or by a module 96, in response to the input for alarm history indicating a display, the alarm history is displayed for a selected patient.

In a decision step or by a module 98, an input for silencing alarm conditions for a selected patient is received and a display is determined. In a step or by a module 100, in response to the input indicating silencing the alarm conditions, the alarm conditions are silenced. The input can be received by voice and/or touch by the input devices. The step can include notifying the central monitoring computing device and/or the monitoring devices of the patient to silence the alarm condition.

In a decision step or by a module 102, the steps can be repeated with additionally received physiological and alarm data.

It is to be appreciated that in connection with the particular illustrative embodiments presented herein certain structural and/or functional features are described as being incorporated in defined elements and/or components. However, it is contemplated that these features may, to the same or similar benefit, also likewise be incorporated in other elements and/or components where appropriate. It is also to be appreciated that different aspects of the exemplary embodiments may be selectively employed as appropriate to achieve other alternate embodiments suited for desired applications, the other alternate embodiments thereby realizing the respective advantages of the aspects incorporated therein.

It is also to be appreciated that particular elements or components described herein may have their functionality suitably implemented via hardware, software, firmware or a combination thereof. Additionally, it is to be appreciated that certain elements described herein as incorporated together may under suitable circumstances be stand-alone elements or otherwise divided. Similarly, a plurality of particular functions described as being carried out by one particular element may be carried out by a plurality of distinct elements acting independently to carry out individual functions, or certain individual functions may be split-up and carried out by a plurality of distinct elements acting in concert. Alternately, some elements or components otherwise described and/or shown herein as distinct from one another may be physically or functionally combined where appropriate.

In short, the present specification has been set forth with reference to preferred embodiments. Obviously, modifications and alterations will occur to others upon reading and understanding the present specification. It is intended that the invention be construed as including all such modifications and alterations insofar as they come within the scope of the appended claims or the equivalents thereof. That is to say, it will be appreciated that various of the above-disclosed and other features and functions, or alternatives thereof, may be desirably combined into many other different systems or applications, and also that various presently unforeseen or unanticipated alternatives, modifications, variations or improvements therein may be subsequently made by those skilled in the art which are similarly intended to be encompassed by the following claims.

What is claimed is:

1. A medical monitoring device, comprising:
a data processor configured to:
construct a shape icon representative of received monitored physiological and alarm data for a plurality of vital signs monitored for at least one patient, the shape icon divided into wedges and each wedge corresponds to one of the plurality of vital signs, measured parameters, or lab test results, and label each shape icon with an identity of one of the at least one patient, and the identity includes at least one of a patient name and a patient location; and
a display device communicatively connected to the data processor and configured to display the constructed shape icon and the identity for the at least one patient.

2. The medical monitoring device according to claim 1, further including:
a wireless communications unit connected to the data processor and configured to receive the monitored physiological and alarm data from a central monitoring computing device.

3. The medical monitoring device according to claim 2, further including:
a frame assembly which mounts the data processor, the display device, and the wireless communication unit to be worn on at least one of a head and a wrist.

4. The medical monitoring device according to claim 1, wherein the data processor is further configured to format each wedge according to an alarm state and the format includes at least one of a color, pattern, and intensity.

5. The medical monitoring device according to claim 4, wherein the format of each wedge includes values of the corresponding vital sign, measured parameter, or lab test result.

6. The medical monitoring device according to claim 4, wherein the format of at least one wedge includes a single indicator located within the at least one wedge indicating one of an increase and a decrease in the vital sign, measured parameter, or lab test result.

7. The medical monitoring device according to claim 1, further including:
a microphone configured to receive a voice input indicating one or more of silencing an alarm, displaying a different patient's shape icon, and showing an alarm history.

8. The medical monitoring device according to claim 1, wherein the shape icon is a circular icon and the plurality of vital signs include non-invasive blood pressure (NBP), heart rate (HR), respiration rate (RR), blood oxygen (SpO₂), BUN, creatine, and electrolytes.

9. The medical monitoring device according to claim 1, wherein the data processor is further configured to construct in the center of the circular icon a concentric circle with an early warning score for the at least one patient.

10. The medical monitoring device according to claim 9, further including:
an audio output device configured to sound at least one of a tone, an intensity, and a pattern indicative of the early warning score for the at least one patient.

11. A method of monitoring physiological and alarm data, comprising:
constructing a shape icon representative of received monitored physiological and alarm data for a plurality of vital signs, measured parameters, or lab test results monitored for at least one patient, the shape icon divided into wedges and each wedge corresponds to one of the plurality of vital signs, measured parameters, or lab test results, and labeling each shape icon with an identity of one of the at least one patient, and the identity includes at least one of a patient name and a patient location; and
displaying the constructed shape icon and the identity for the at least one patient on a display device.

12. The method of monitoring physiological and alarm data according to claim 11, further including:
receiving wirelessly the monitored physiological and alarm data from a central monitoring system.

13. The method of monitoring physiological and alarm data according to claim 11, wherein constructing the shape icon includes:
formatting each wedge according to an alarm state which includes at least one of a color, pattern, and intensity

14. The method of monitoring physiological and alarm data according to claim 11, further including:
displaying an alarm history for the at least one patient, which includes for each alarm: a time and values triggering the alarm.

15. The method of monitoring physiological and alarm data according to claim 11, further including:
receiving input indicating silencing of alarms for the at least one patient; and
silencing the alarms for the at least one patient.

16. The method of monitoring physiological and alarm data according to claim 11, wherein the shape icon is circular and constructing the circular icon includes:
constructing in the center of the circular icon a concentric circle with an early warning score for the at least one patient.

17. The method of monitoring physiological and alarm data according to claim 16, further including:
sounding at least one of a tone, an intensity, and a pattern indicative of the early warning score for the at least one patient.

18. A non-transitory computer-readable storage medium carrying software which controls one or more electronic data processing devices to perform the method according to claim 11.

19. An electronic data processing device configured to perform the method according to claim 11.

20. A medical monitoring device, comprising:
a wireless communication unit configured to receive the monitored physiological and alarm data from a central patient monitoring system;
a data processor communicatively connected to the wireless communication unit configured to:
construct a pie shaped representation of the received monitored physiological and alarm data for a plurality of vital signs, measured parameters, or lab test results, monitored for at least one patient, the pie shaped representation divided into wedges and each wedge corresponds to one of the plurality of vital signs, measured parameters, or lab test results;
construct a concentric circle in the pie shaped representation and the concentric circle includes an early warning score for the at least one patient; and
label each circular icon with an identity of one of the at least one patient, and the identity includes a patient name and a patient location; and
a display device communicatively connected to the data processor and configured to display the constructed circular icon and the identity for the at least one patient.
